SEQUENCE LISTING (8 sequences)

SEQ ID NO: 1 - DNA, 21 bp, Artificial Sequence
  Description of Artificial Sequence primer_bind
catgacaaggcctgcgtccga

SEQ ID NO: 2 - DNA, 21 bp, Artificial Sequence
  Description of Artificial Sequence primer_bind
gccgcctccacctgtagaaca

SEQ ID NO: 3 - DNA, 33 bp, Artificial Sequence
  Description of Artificial Sequence primer_bind
ttggacacactgcagctggacgtcgccgacttt

SEQ ID NO: 4 - DNA, 33 bp, Artificial Sequence
  Description of Artificial Sequence primer_bind
attgcagagccagggctggggagcagtcatagt

SEQ ID NO: 5 - DNA, 23 bp, Artificial Sequence
  Description of Artificial Sequence primer_bind
tcagcaagaactgcaacaacagc

SEQ ID NO: 6 - DNA, 20 bp, Artificial Sequence
  Description of Artificial Sequence primer_bind
gtgaggaagatccagggcga

SEQ ID NO: 7 - DNA, 270 bp, Homo sapiens
actctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcatgtggtgattccccggatccaccccaactccatctgtgcagcaaacaa

SEQ ID NO: 8 - protein, 90 aa, Homo sapiens
LCLVPLSLAQIDLNITCRFAGVFHVEKNGRYSISRTEAADLCKAFNSTLPTMAQMEKALSIGFETCRYGFIEGHVVIPRIHPNSICAANN

(19) United States
(12) Patent Application Publication
(10) Pub. No.: US 2004/0204384 A1
(43) Pub. Date: Oct. 14, 2004

(54) Methods for regulating hematopoietic cell differentiation with polymers containing disaccharide units

(75) Inventors: Florence Smadja-Joffe, Fontenay aux Roses (FR); Rachida-Sihem Charrad, Villejuif (FR); Christine Chomienne, Paris (FR); Bertrand Delpech, Saint Aubin Les Elbeuf (FR); Claude Jasmin, Charenton (FR)

Correspondence Address: NIXON & VANDERHYE, PC, 1100 N GLEBE ROAD, 8TH FLOOR, ARLINGTON, VA, 22201-4714, US

(73) Assignee: Institut National De La Sante Et De La Recherche Medial (Inserm), Paris (FR)

(21) Appl. No.: 10/837,671

(22) Filed: May 4, 2004

Related U.S. Application Data
(62) Division of application No. 09/927,463, filed on Aug. 13, 2001.
Application No. 09/927,463 is a continuation of application No. PCT/FR00/00349, filed on Feb. 11, 2000.

(30) Foreign Application Priority Data
Feb. 11, 1999 (FR) 99 01644

(51) Int. Cl. A61K 31/715

(52) U.S. Cl. 514/54

(57) ABSTRACT

The invention concerns the use of a polymer comprising an efficient amount of

disaccharide units each consisting of a molecule with N-acetyl-D-glucosamine structure bound by the O-glucoside β1,4 linkage to a molecule with glucuronic acid structure for producing a medicine designed to induce or stimulate the differentiation of hematopoietic cells, and leukemic cells in particular.

This application is a divisional of application Ser. No. 09/927,463, filed Aug. 13, 2001, which is a continuation of PCT/FR00/00349, filed Feb. 11, 2000, the entire contents of which are hereby incorporated by reference.

The present invention, as a general rule, relates to means used to regulate the differentiation of haematopoietic cells. Remarkably, the regulation means according to the invention apply to the differentiation of cells wherein the differentiation no longer corresponds to a normal profile and particularly to cells wherein differentiation is inhibited (leukaemic cells, in particular acute myeloblastic leukaemia blasts). According to another remarkable aspect, the regulation means according to the invention also apply to the differentiation of haematopoietic cells. Indeed, the regulation means according to the invention make it possible to induce or stimulate the differentiation of leukaemic cells and AML blasts in particular, and that of strain cells according to the granulocytic process and the monocytic process.

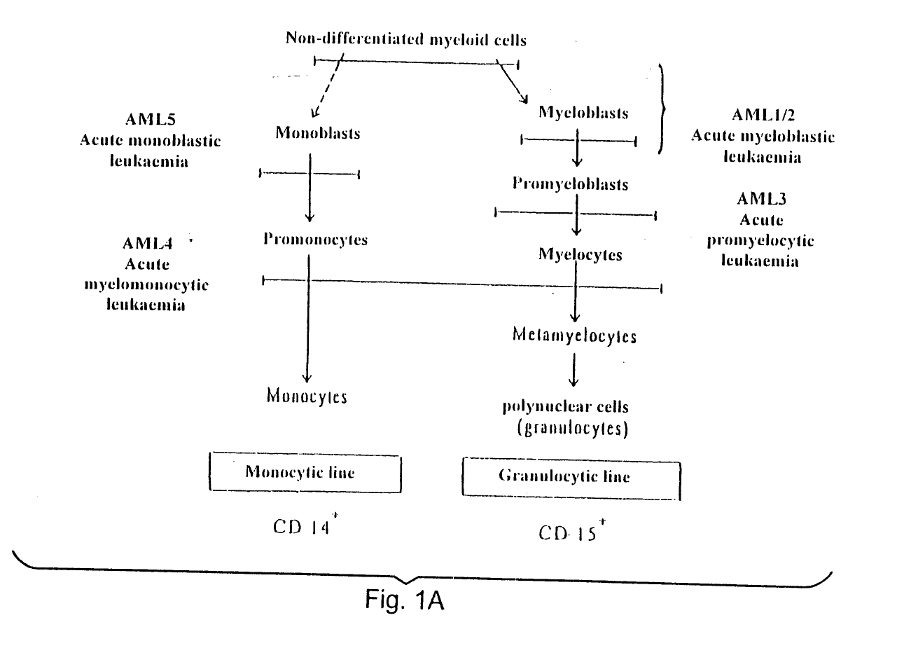

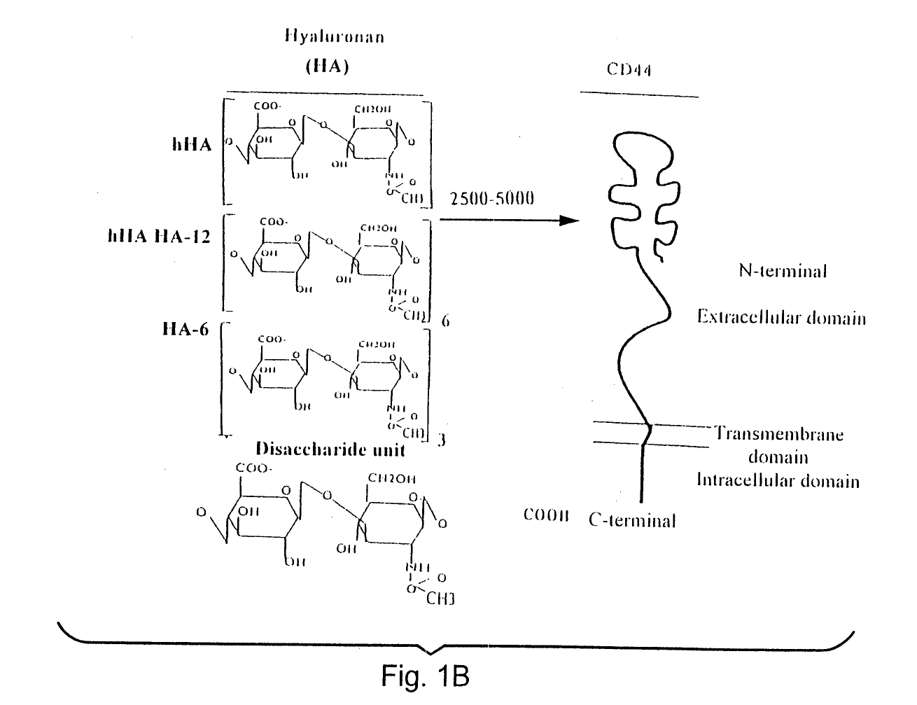

Different types of leukaemia may be identified: lymphoblastic leukaemias, which particularly comprise acute lymphoblastic leukaemias (ALL) or lymphomas and myeloblastic leukaemias which particularly comprise acute myeloblastic leukaemias (AML). AML represents approximately half of the cases of leukaemia, i.e. approximately 1000 new cases a year in France and 6500 in the USA, with an incidence which increases exponentially over 40 years. AML corresponds to an inhibition of the differentiation of myeloid cells at an immature stage and is conveyed by invasion of the bone marrow and circulating blood by blastic cells, the cytological characteristics of which define the different AML sub-types classified M1 to M7 (French-American-British (FAB) classification), the most frequent being types M1 to M5 (see FIG. 1A).

In spite of spectacular therapeutic progress in recent years, AML remains a severe disease since the first remission, although it can be induced in 70% of cases, frequently does not last for more than one year, and 60% of patients relapse within 5 years. AML relapse treatments, which generally require bone marrow grafts, are experiencing significant limitations due to the rarity of related donors and an age limit of 45 years.

Recently, the induction of the differentiation of leukaemic blasts in mature granulocytes by administering retinoic acid (RA, or all-transretinoic acid ATRA) has improved the clinical progression of patients suffering from M3 AML spectacularly, wherein the remission rate is currently 70% after 5 years. However, this differentiation treatment is not applicable to patients suffering from M3 sub-type AML, a rare sub-type which only represents approximately 10% of AML cases, and poses in vivo resistance problems. In addition, this treatment remains ineffective for other types of AML.

The present invention provides means to regulate the differentiation of haematopoietic cells which, in a particularly remarkable manner, may be applied to cases of cells wherein differentiation is inhibited, such as leukaemic cells, and, remarkably, AML blasts. Indeed, the regulation means according to the invention make it possible to induce or stimulate the differentiation of leukaemic cells, particularly AML cells, and are, remarkably:

effective on leukaemic blasts directly from patients (particularly AML blasts): they are effective not only against model cell lines, i.e. study lines designed to be able to self-proliferate easily in vitro, but, remarkably, also are effective against leukaemic cells directly from patients, i.e. cells which show little or no ability to divide in vitro, and wherein the survival in such cultures is limited over time (generally less than one week), and

effective against not only one but several AML sub-types: they particularly make it possible to induce the differentiation of M1/2, M3, M4, M5 AML blasts, which are the most frequent sub-types. In addition, it is not excluded that they may be used against less frequent AML sub-types, and AML6 and/or AML7 in particular.

Therefore, the regulation means according to the invention are effective against different AML sub-types, including against sub-types for which no effective differentiating treatment had yet been produced (sub-types M1/2, M4, M5). Indeed, they make it possible not only to induce differentiation according to the granulocytic process of inhibited blasts at the M3 stage, but also make it possible 1) to stimulate differentiation according to the granulocytic and monocytic processes of inhibited blasts at a very immature stage (M1/M2) and inhibited blasts at the M4 stage, at 2) to induce differentiation according to the monocytic process of inhibited blasts at the M5 stage.

Advantageously, the regulation means according to the invention make it possible not only to induce or stimulate the differentiation of leukaemic cells directly from patients, and AML cells in particular, but may also be used to inhibit the in vivo proliferation of such leukaemic cells.

The means according to the invention are also effective in regulating the differentiation of very immature (not completely differentiated) normal haematopoietic cells, particularly that of strain cells comprising no differentiation antigen such as CD14⁻ CD15⁻ normal haematopoietic cells (CD34⁺, or CD34⁻ cells). They are used to differentiate not only according to the monocytic process, but also according to the granulocytic process, and are effective on cells from patients, and also in vivo.

The regulation means according to the invention also offer the benefit of showing no or a low potential toxicity for the patient, up to doses of several mg, which represents a major advantage for the patient, and which enables the use of the product at effective doses.

In this way, the present invention relates to a medicinal product intended to induce or stimulate the differentiation of cells selected from the group composed of leukaemic cells and CD14⁻ CD15⁻ strain cells, characterised in that it comprises at least one polymer comprising an effective quantity of disaccharide units each composed of an N-acetyl-D-glucosamine structure molecule bonded by an O-glycoside β1,4 bond with a glucuronic acid structure molecule, and the use of such a polymer for the production of a medicinal product intended to induce or stimulate the differentiation of such cells. A representation of such a disaccharide unit is given in FIG. 1B. It may be noted that the term “polymer” covers, in the present application, both oligomers and polymers, and the terms “medicinal product” and “treatment” cover, in the present application, any form of control of a given pathological or undesired condition, including therapy, the prevention of worsening of the pathological condition, the palliation or alleviation of the patient's living conditions. Advantageously, the use of said polymer according to the invention enables the production of a medicinal product, which, in addition to its abilities to induce or stimulate the differentiation of leukaemic cells, may be used to inhibit the proliferation of such leukaemic cells.

The term “effective quantity” is used in the present application to refer to a number of disaccharide units enabling the resulting polymer to induce or stimulate the differentiation of the targeted leukaemic cells significantly. Examples of means used to test whether a polymer contains a suitable number of disaccharide units comprise placing this polymer into contact with the targeted leukaemic cells, and particularly with such cells sampled from humans, under physiological conditions. Examples of such placing in contact are known to those skilled in the art, some of which are given in the “examples” section below. Briefly, the term physiological conditions is used in the present application to refer to in vivo conditions or in vitro condition imitating in vivo conditions in an optimal manner (in the case of a medicinal product for example: medium used for the culture of targeted leukaemic cells such as 10% serum RPMI 1640 (foetal calf serum FCS or autologous serum), suitable temperature for the cell cultures in question, i.e. generally of the order of approximately 37° C., humidity-saturated atmosphere containing air and CO₂ in suitable proportions for the cell cultures in question). The term leukaemic cells sampled from humans is used in the present application to refer to freshly sampled cells, and/or cells having been preserved by freezing after sampling. Such leukaemic cells may particularly be obtained by sampling blood or sampling bone marrow cells, and then recovering the white blood cells, with elimination of lymphocytes if required. The term CD14⁻ CD15⁻ strain cells particularly refers to normal haematopoietic cells capable of progeny and self-replication, which comprise no differentiation antigen such as CD14, CD15, i.e. CD14⁻ CD15⁻ strain cells which are CD34⁺ or CD34⁻.

Advantageously, an effective quantity of said saccharide units according to the invention is equivalent to a number of disaccharide units greater than or equal to approximately 3. Below 3 units, the efficacy of the use according to the invention appears to be significantly less industrially profitable. A polymer complying with such characteristics particularly corresponds to hyaluronic acid (HA), a large molecule with 2500-5000 disaccharide units (formula GlcAU(β1-3)-[GlcNAc(β1-4)GlcAU(β1-3)]ₙ-GlcNAc), or to an HA fragment comprising at least three disaccharide units (from HA-6). Preferentially, said effective quantity is equivalent to a number of disaccharide units approximately between 3 and 10 (including terminals). Therefore, a preferred use according to the invention comprises the use for the production of said medicinal product of HA fragments containing at least 3 and at most 10 disaccharide units approximately (approximately from HA-6 to HA-20): HA-6 and/HA-12, for example. Depending on the desired effect, polymers comprising more than 10 said disaccharide units also offer products of interest. In particular, polymers comprising from 10 to approximately 100 disaccharide units are also effective. The use of fragments comprising a small number of disaccharide units is preferred for simple reasons relating to easy production. Those skilled in the art will be able to optimise the choice of a number disaccharide units and the choice of effective quantities.

Those skilled in the art may avail of several sources of a suitable polymer for the use according to the invention: they may for example be extracted from natural sources (for HA: human umbilical cord, streptococcus, cockscomb, in particular) and subjected if required to enzyme digestion (see “examples” section below, HA digestion by hyaluronidase), and/or directly purchased from suppliers such as ICN Pharmaceuticals, Sigma (e.g. HA, HA fragments). Said polymer may also be used in salt form, such as sodium or potassium hyaluronate in powder form, or dissolved in saline solution. Such a polymer may also comprise chemical modifications, particularly so as to modulate the specificity of said polymer with respect to the target leukaemic cells (particularly AML cells), modulate its lifetime and/or its bioavailability.

A use according to the invention may particularly comprise a use of said polymer for the production of a medicinal product wherein the unit dose is between approximately 1 and 10 mg/kg inclusive, advantageously between approximately 2 and 5 mg/kg, particularly of the order of approximately 3 mg/kg. This dose may be increased or reduced (unit dose) and/or repeated (over time) to optimise the efficacy of the product. Since the polymer used according to the invention is not generally toxic, its dosage may be adapted to the patient in question, according for example to the disease follow-up results. The invention provides, for this purpose, an in vitro method which makes it possible to predict, for a given patient, the therapeutic efficacy, particularly against leukaemia, of a medicinal product obtained by means of a use according to the invention. The in vitro prediction method according to the invention particularly comprises:

placing in contact, under physiological conditions (for example, approximately 37° C., medium suitable for the targeted cell culture such as 10% serum (foetal calf serum FCS or autologous serum) RPMI 1640, humidity-saturated atmosphere containing air and CO₂ in suitable proportions), of the medicinal product under test with characteristic cells of the pathological or undesired condition in question, from the given patient, and in the case of a leukaemic patient, with leukaemic blasts from said patient,

in vitro observation of the existence or the significant absence of the desired therapeutic effect with reference to the negative control, and in the case of a leukaemic patient, observation of the existence or absence of at least one significant differentiating effect on said cells with reference to the negative control (see below and in the “examples” section for illustrations of such effects),

prediction of good therapeutic efficacy (particularly against leukaemia) in vivo of the medicinal product under test for the patient in question if said therapeutic effect(s), particularly differentiating effect(s) is/are observed as being present in vitro.

An experimental animal model may also be used.

Such prediction methods according to the invention represent a good tool to adapt the administration dosage (unit dose, frequency) of a medicinal product according to the invention.

According to an alternative embodiment, a use according to the invention may comprise the use of a mimetic agent of said polymer (mimetic of hyaluronic acid or a fragment of this acid in particular), and of an agonist agent, in particular, such as that obtained by screening in a chemical and/or biological bank. Means to carry out such screenings or selections are known to those skilled in the art: they may be in particular by carried out by means of functional and/or differential screening, flow cytometry, for example (selection of compounds capable of bonding with the same cell targets as said polymer and with CD44 in particular and capable of producing at least an equivalent type of differentiating and/or anti-proliferation effect). Such a mimetic or agonist agent may be used, according to the invention, as an alternative to the use of said polymer presented above, or in addition to this use. Advantageously, those mimetics or agonists which are not toxic for humans, and/or which are not liable to induce undesired antigenic reactions may be used. In this way, if the screened bank is an antibody bank (particularly monoclonal antibodies), human (monoclonal) or humanised antibodies (see examples) may advantageously be chosen for the production of said medicinal product.

A use according to the invention may also comprise, in addition to said polymer or mimetic, other active agents for the induction and/or stimulation of the differentiation of haematopoietic cells, and/or leukaemic cells, and/or AML cells in particular, such as cytokines for example. Said other active agents may be incorporated in said medicinal product, or be administered in parallel with said medicinal product and presented in the form of a kit comprising, firstly, said other agents, and, secondly, said polymer. Therefore, said polymer or mimetic is, in the medicinal product according to the invention, an active agent which may be used as an active co-agent.

Said use may also comprise, in addition to the use of said polymer or mimetic, the use of an adjuvant compound capable of stimulating the bonding of said polymer with its cell target, such as an anti-CD44 antibody capable of stimulating such a bond, or a fragment (Fab, (Fab′)₂, Fv, CDR) of such an antibody. In relation to this aspect, it may be underlined that any product in general, and antibody in particular, considered as activating a cell target such as CD44 does not necessarily represent a product with a differentiating and/or anti-proliferation activity by means of bonding with its target and with CD44 in particular, and that all products (particularly antibodies) considered as having a differentiating and/or anti-proliferation activity by means of bonding with its target such as CD44 do not necessarily represent a product capable of stimulating the bonding of said polymer or mimetic with said target: some anti-CD44 differentiating antibodies block and inhibit the bonding of HA with CD44 (see “examples” section). However, those skilled in the art may avail of means (flow cytometry, for example) used to determined whether a compound is or is not capable of stimulating, in a satisfactory manner for the target applications, the bonding of said polymer or mimetic with its cell target and with CD44 in particular.

A use of said polymer according to the invention enables the production of said medicinal production in any suitable pharmaceutical formulation for the desired administration, and particularly in the form of tablets, granules, capsules, powder forms, suspensions, oral solutions, solutions for injection or patches. This technical adaptability represents a considerable advantage of the use according to the invention. A use of said polymer according to the invention enables the production of solution, particularly saline solution. Such manufactures are produced under conditions adapted to the physico-chemical properties of the polymer or mimetic used, particularly to pH conditions, at adapted concentrations.

A use according to the invention may comprise, in addition to the use of said polymer or mimetic, the use of any suitable compound or excipient for the desired pharmaceutical formulation, particularly any suitable pharmaceutically inert vehicle. A solution for injection, particularly by the intravenous route, appears to be an easy to produce formulation of interest, said polymer being freely soluble in equilibrated saline solution. This solubility represents an advantage over ATRA, which is not soluble in saline solution.

Advantageously, a use according to the invention results in a medicinal product wherein the placing in contact, under physiological conditions, with a statistically representative number of leukaemic cell (blasts) samples, taken from humans is conveyed by, on said cells, at least one significant differentiating effect such as the induction or stimulation of nitroblue tetrazolium reduction, and/or an increased expression of specific haematopoietic cells in maturation such as CD14 (monocytic process) or CD15 (granulocytic process) and/or the induction or stimulation of specific cytological characteristics of haematopoietic cells in maturation (such as a reduction in the nucleus/cytoplasm ratio, reduction in the number of nucleoles, chromatin condensation, nuclear segmentation, restricted number of azurophilic granulations, irregular cytoplasmic contours). Other significant differentiating effects comprise a molecular event marking a haematopoietic differentiation such as PML-RARα oncoprotein degradation, and/or an induction or stimulation of intracellular tyrosine phosphorylations, and/or an induction or stimulation of at least on differentiation factor messenger such as a differentiating cytokine (e.g. G-CSF, M-CSF). Advantageously, the placing in contact under physiological conditions of a medicinal product according to the invention with a statistically representative number of leukaemic cell (blasts) samples taken from humans is also conveyed by, on said cells, a significant inhibition of their proliferation. Means to produce such placing in contact, such physiological conditions, and such leukaemic cells are known to those skilled in the art, examples of which have been given above and are also presented in the “examples” section below. The term statistically representative number of samples, in the present application, refers to a number of samples enabling a valid statistical analysis of the results, particularly a number greater than approximately 10 samples, for example of the order of approximately 20-30 samples. The term significant effect refers to a statistically significant average effect with reference to the negative controls: for example, an effect which is not significantly observed, on average, in the negative controls, and which is significantly observed, on average, in at least approximately 75% of the test samples.

The role of said polymer contained in the medicinal product according to the invention is a direct role. Indeed, said polymer acts by bonding with a molecule on the surface of said cells, which then acts as a transducing receptor of a pro-differentiation and/or anti-proliferation signal. Said polymer is capable of showing this activity in the absence of any other differentiating product. For example, it is capable of exerting its differentiating action in vitro in a serum-based medium (foetal calf serum (FCS) or autologous serum) with no added cytokine. In the presence of normal haematopoietic cells, such as progenitor CD34⁺ cells, it is capable of exerting its differentiating action in vitro in a serum-free medium. Means to observe such capabilities are known to those skilled in the art. Examples are given in the “examples” section below.

This activity of said polymer on haematopoietic cells is particularly exerted by activating the CD44 receptor. It is not excluded that it can exert (independently or in conjunction) via any other membrane receptor capable of fixing said polymer (HA, HA fragment, for example) and inducing a differentiating signal on the cell expressing this receptor. Such a potential receptor may be advantageously chosen from the molecules of the hyaladherin family (RHAMM, hyaluronectin). Those skilled in the art may avail of numerous means to test whether a potential receptor can be recognised by said polymer, and whether this receptor then transduces a cellular differentiation signal. Such means particularly comprise:

i. detection, using flow cytometry techniques, for example, of a bond between said polymer (HA and/or HA fragments containing at least 3 disaccharide units, in particular) with cells expressing said potential receptor and an absence of a bond between the same polymer(s) and the same cells when the access to said potential receptor is specifically inhibited, and/or,

ii. detection of at least one differentiating effect on cells expressing the potential receptor placed in the presence of said polymer (HA and/or HA fragments containing at least 3 disaccharide units, in particular), by comparing with the same type of cells placed under equivalent conditions but in the absence of the same polymer(s). Examples of such differentiating and/or anti-proliferation effects may be found below in the “examples” section, in particular.

However, in order to prevent any undesired bonding of said polymer with molecules on the surface of non-targeted cells, for example any bonding of said polymer (HA and/or fragments) with the ICAM1 receptor on the liver sinuses, a use according to the invention may also comprise the use of compounds inhibiting said non-targeted molecules, for example, anti-ICAM1 compounds inhibiting the bonding of said polymer with ICAM1, such as anti-ICAM1 monoclonal antibodies, or chondroitin sulphate which, by bonding with ICAM1, blocks the accessibility of ICAM1 to HA. According to another embodiment of the invention, a compound capable of preventing bonding of said polymer or mimetic molecule with an undesired cell target may also be used. Examples of such compounds comprise anti-ICAM1 monoclonal antibodies, or a fragment (Fab, (Fab′) Fv, CDR) of such antibodies.

According to an advantageous embodiment of the invention, said leukaemic cells are myeloblastic leukaemia cells (blasts), acute myeloblastic leukaemia cells in particular. They may particularly consist of AML1/2 and/or AML3 and/or AML4 and/or AML5 blasts. The use according to the invention is advantageously intended for the production of an anti-myeloblastic leukaemia medicinal product, and anti-AML1/2 and/or anti-AML3 and/or anti-AML4 and/or anti-AML5 and/or AML6 and/or anti-AML7 in particular.

Therefore, as specified above, and as illustrated in the examples, the use of said polymer according to the invention enables the production of a medicinal product which is intended to stimulate or induce the differentiation of cells wherein the differentiation is inhibited, particularly leukaemic cells, and remarkably, AML blasts. Said medicinal product obtained in this way according to the invention is nonetheless capable, under physiological conditions, of stimulating or inducing the differentiation of normal (not completely differentiated) haematopoietic strain cells, without inhibiting their proliferation: indeed, it may stimulate or induce the differentiation of healthy human haematopoietic strain cells (CD14⁻ CD15⁻ CD34⁺, or CD34⁻). Consequently, the use according to the invention thus makes it possible to produce a medicinal product which can be administered to patients diagnosed with leukaemia in order to stimulate or induce the differentiation of their normal human strain cells. Similarly, it makes it possible to produce a medicinal product which can be administered to non-leukaemic patients in order to stimulate or induce the differentiation of their normal (not completely differentiated) haematopoietic cells, particularly in order to treat aplasia, or neutropenia. Therefore, the present invention relates, as a general rule, to a medicinal product intended to induce or stimulate the differentiation of CD14⁻ CD15⁻ strain cells or leukaemic cells, and AML blasts in particular, characterised in that it comprises an effective quantity of disaccharide units each composed of an N-acetyl-D-glucosamine structure molecule bonded by an O-glycoside β1,4 bond with a glucuronic acid structure molecule and also relates to the use of such an effective quantity for the production of such a medicinal product.

The present invention is illustrated by the following examples, given for purely illustrative purposes, which are in no way restrictive. The present invention also comprises any alternative embodiment that may be produced by those skilled in the art, without undue experimentation, from the disclosure given by the present application (including disclosure, examples, claims and figures) and means according to the prior art.

The “examples” section below refers to the following figures:

in FIG. 1A, the most frequent AML sub-types (FAB classification: M1/M2 AML or AML1/2, M3 AML or AML3, M4 AML or AML4, M5 AML or AML5) are indicated for the myeloid differentiation stage at the inhibition to which they correspond,

FIG. 1B gives a schematic representation of the CD44 cell surface molecule and hyaluronic acid (HA) molecules, which are capable of bonding with CD44: human hyaluronic acid hHA containing 2500-500 disaccharide units, hHA fragment containing 6 disaccharide units (HA-12) and hHA fragment containing 3 disaccharide units (HA-6).

FIGS. 2A-1, 2A-2, 2A-3, 2A-4 and FIGS. 2B-1, 2B-2, 2B-3, 2B-4, 2B-5 and 2B-6 illustrate the fact that hyaluronic acid (HA) is capable of inducing the differentiation of all AML blast sub-types:

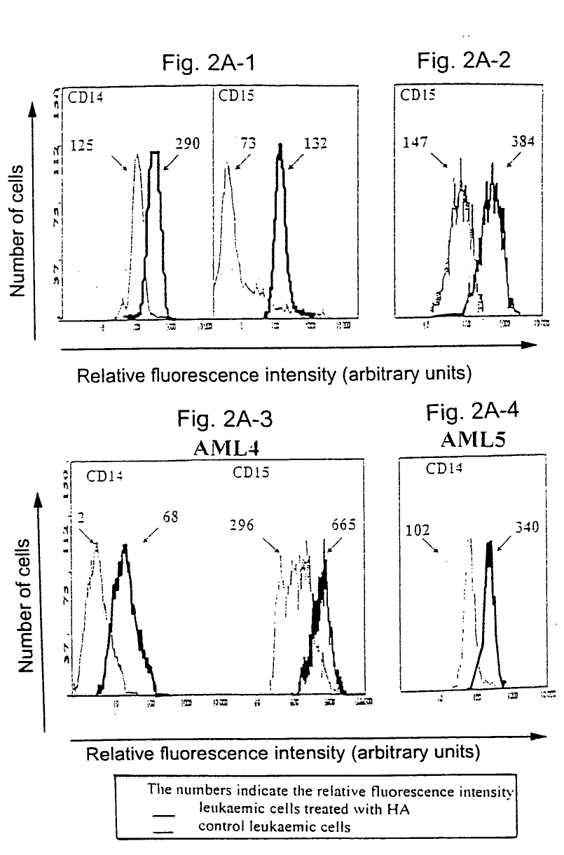

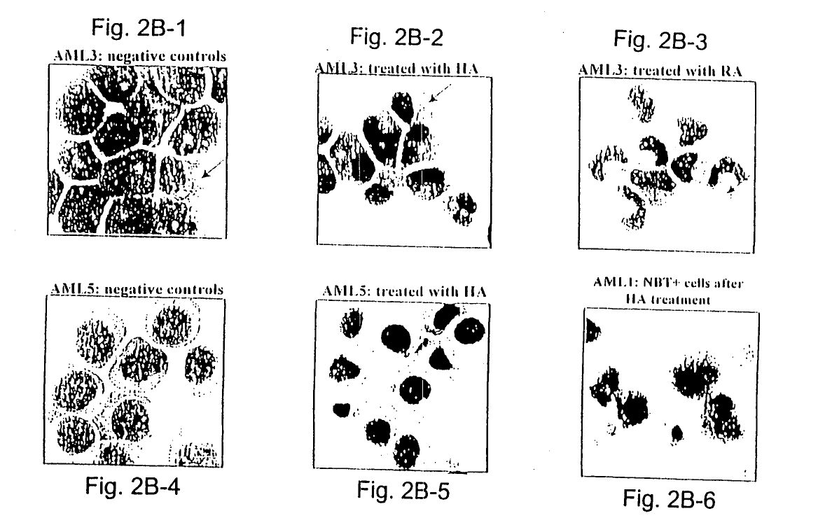

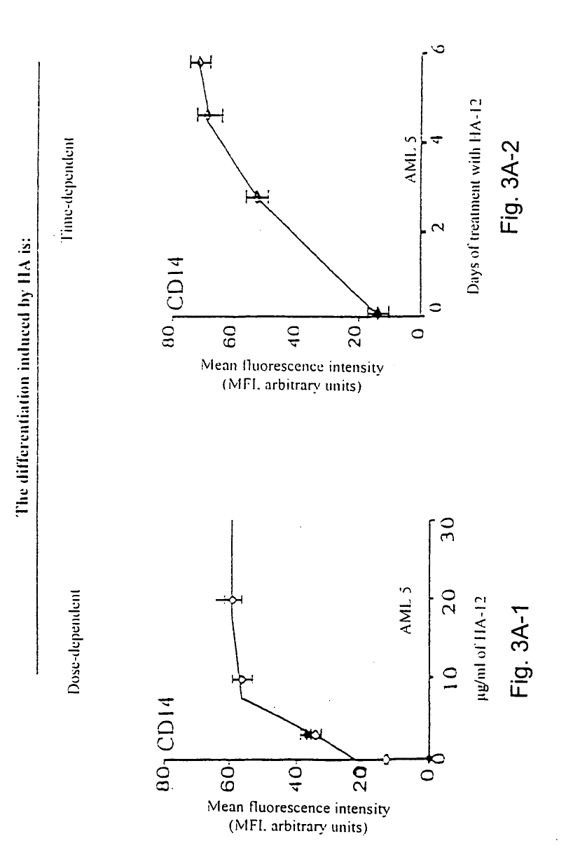

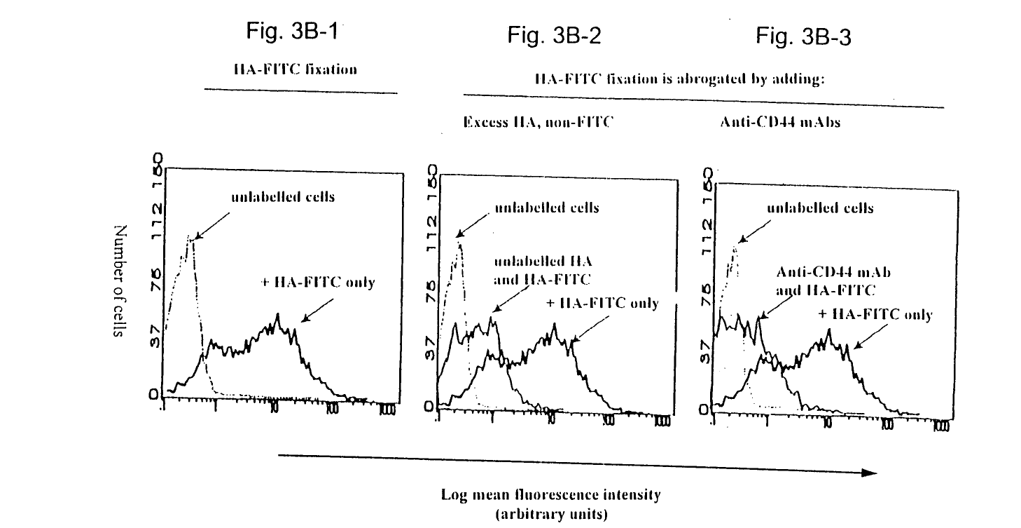

FIGS. 2A-1, 2A-2, 2A-3 and 2A-4: graphs representing the number of CD14⁺ and CD15⁺ cells induced by using HA on AML1/2 (FIG. 2A-1), 3 (FIG. 2A-2), 4 (FIG. 2A-3) and 5 (FIG. 2A-4), as a function of the fluorescence intensity (black curves: use of HA; grey curves to the left: negative controls),

FIGS. 2B-1, 2B-2, 2B-3, 2B-4, 2B-5 and 2B-6: photos illustrating induction on AML blasts by HA (CD44 activation of specific cytological characteristics for mature cells (FIG. 2B-1 for AML3: negative control, FIG. 2B-2 for AML3: treated with HA, FIG. 2B-3 for AML3: treated with RA, FIG. 2B-4 for AML5: negative controls, FIG. 2B-5 for AML5: treated with HA, and FIG. 2B-6 for AML1: NBT+ cells after HA treatment),

FIGS. 3A-1, 3A-2 and 3B-1, 3B-2 and 3B-3 illustrate the dose-dependent and time-dependent nature of the differentiation induced by hyaluronic acid (HA):

FIGS. 3A-1 and 3A-2: mean fluorescence intensity (CD14 MFI) measured on AML5 blasts as a function of the HA-12 dose (μg/ml) (graph on left (FIG. 3A-1)) and as a function of incubation time in the presence of HA-12 (graph on right (FIG. 3A-2)),

FIGS. 3B-1, 3B-2 and 3B-3: HA-FITC bonding and inhibition of this bonding, as illustrated by the number of cells as a function of the log of the fluorescence intensity for (curves identified from left to right for each graph):

graph on left (FIG. 3B-1): unlabelled cells, and AML blasts incubated with HA-FITC only,

centre graph (FIG. 3B-2): unlabelled cells, AML blasts incubated with unlabelled HA and with HA-FITC, AML blasts incubated with HA-FITC only,

graph on right (FIG. 3B-3): unlabelled cells, AML blasts incubated with anti-CD44 monoclonal antibodies (mAb), and with HA-FITC, AML blasts incubated with HA-FITC only,

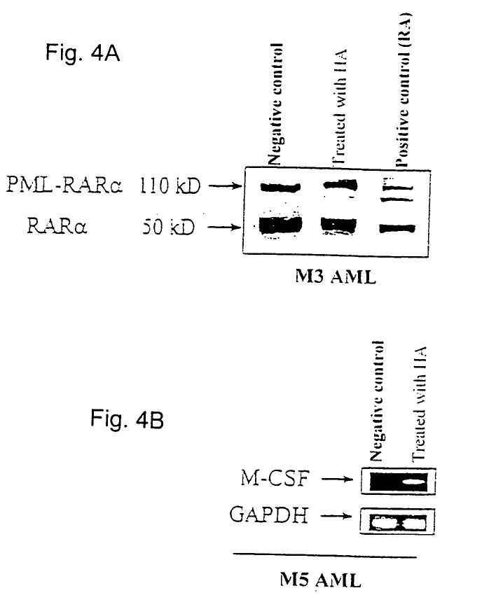

FIGS. 4A and 4B illustrate the molecular events marking the induction by HA (CD44 activation) of AML blast differentiation:

FIG. 4A: degradation of PML-RARα oncoprotein (top lines at 110 kDa) in M3 AML blasts, 24 hours after treatment with HA, and maintenance of the RARα wild protein (bottom lines at approximately 50 kD), as detected using the ECL chemoluminescent system (track 1: negative control, track 2: blasts treated with HA, track 3: positive control (blasts treated with RA)),

FIG. 4B: induction by HA of M-CSF transcript synthesis in M5 AML blasts (two grouped photos: M-CSF at top of this group, GADPH marker at bottom), as displayed by electrophoresis of total RNA on agarose gel and specific hybridisations (for each gel: track 1=negative control, track 2: blasts treated with HA),

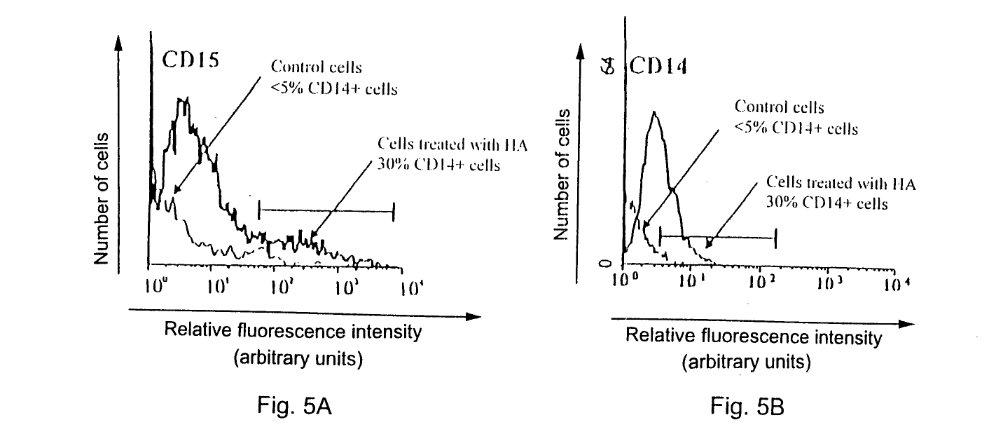

FIGS. 5A and 5B: induction by HA fragments of very immature haematopoietic cell differentiation (CD34⁺ CD14⁻ CD15⁻ strain cells) according to the monocytic process and according to the granulocytic process.

### EXAMPLE 1

### Induction, by HA and Particularly Via CD44, of AML Blast Differentiation

### Materials and Methods

AML Patients.

Leukaemic peripheral blood or bone marrow samples were taken at the time of diagnosis, after informed consent, from 36 patients suffering from acute myeloid leukaemia (AML). The diagnosis of the disease and its classification comply with French-American-British (FAB) classification criteria. All the patients showed more than 60% blasts in the peripheral blood.

AML Blast Separation.

Fresh or frozen AML cells were enriched by centrifugation according to the density gradient in the presence of Ficoll, and washed in RPMI 1640 medium containing 10% foetal calf serum (FCS). The frozen cells were thawed at ambient temperature in RPMI 1640 medium containing 50% FCS, and then washed twice in RPMI medium supplemented with 10% FCS. B and T lymphocytes were eliminated from all the samples, along with monocytes for the AML1/2 and AML3 samples. This elimination was performed by specific immunoadsorption of Dynabeads beads (Dynal, Oslo, Norway) coated with monoclonal antibodies directed against the specific surface antigens CD2 and CD19 (lymphocytes) and CD14 (monocytes), according to the manufacturer's instructions. In this way, cell suspensions containing more than 95% AML blasts were obtained.

Anti-CD44 Monoclonal Antibodies (mAb).

Different anti-CD44 mAb were used in the differentiation induction tests: FIO-44-2 (IgG2a, Serotec, Kidlington Oxford, UK), and Hermes-1 (IgG2a, hybridoma available from Developmental Studies Hybridoma Bank, Iowa), in particular. These mAbs are both capable of transmitting an activating signal. For the negative controls, these mAbs were replaced by murine IgG (non-anti-CD44) of the same isotype.

Different anti-CD44 monoclonal antibodies were used for the HA-FITC bonding tests with CD44: in particular, the mAb J173 (Coulter-Immunotech, Marseille-Luminy, France).

The mAb 6D12 (IgG1, Coulter-Immunotech) was used in the protein phosphorylation tests on tyrosine residue.

Hyaluronic Acid (HA) and Controls.

Hyaluronic acid from human umbilical cords (hHA, ref. 362421) was obtained from ICN Pharmaceuticals (Costa Mesa, Calif.), dissolved at 5 mg/ml in distilled water and boiled for 10 minutes. Several molecular forms of hyaluronic acid (HA) were used: human hyaluronic acid hHA, (high molecular weight form (500 to 2000 kDa) and two types of oligosaccharide fragments HA-6 and HA-12, obtained after digestion of hHA by hyaluronidase (Sigma, St Louis, Mo.), at 37° C. for 6 hours, and isolation using conventional techniques on a chromatography column on AcA202 gel (Biosepara, Villeneuve La Garenne, France). hHA, HA-6 and HA-12 are composed of 2.10³-10⁴, 6 and 12 saccharide units, respectively. All the hyaluronic acid preparations are endotoxin-free. FIG. 1B gives a schematic representation of the human hyaluronic acid molecule (hHA, 2500-5000 disaccharide units), hHA fragment containing 6 disaccharide units (HA-12), hHA fragment containing 3 disaccharide units (HA-6), and a schematic representation of the CD44 cell surface molecule, with which HA is capable of bonding. Each disaccharide unit is composed of a D-glucuronic acid molecule bonded with an N-acetyl-D-glucosamine molecule. Hyaluronic acid bonds with the CD44 molecule in a region located at the N-terminal of the extracellular domain.

As negative controls, the AML blasts were cultured in the presence of chondroitin sulphate (Sigma), a sulphated glycosaminoglycan with a structure similar to that of hyaluronic acid and which is liable to bond with CD44. In addition, leukaemic blasts from AML3 were treated with all-transretinoic acid (RA) as positive differentiation controls for this AML sub-type.

AML blast treatment with HA. Cell suspensions containing more than 95% AML blasts were deposited in triplicate at a rate of 2.105 cells per ml of RPMI 1640/10% FCS in tissue culture plates (Costar Corp., Cambridge, Mass.) with 96 wells each containing 150 μl of medium, and placed in incubation for 5 days in the presence of 20 μg/ml of hyaluronic acid (hHA, HA-6 or HA-12). HA was added at the specified concentrations and chondroitin sulphate was added to the negative controls. The plates were placed in an incubator at 37° C. in a humid atmosphere for 6 days, and the cells underwent the differentiation studies as described below.

Evaluation of Myeloid Differentiation.

Differentiation was detected by analysing 3 criteria: 1) the ability to produce an oxidation-reduction reaction in response to a phorbol ester: this oxidation-reduction reaction is detected with nitroblue tetrazolium NBT reduction, 2) the expression of specific antibodies for differentiation (specific CD14 for monocytes, and specific CD15 for granulocytes), and 3) specific cytological modifications. All these criteria are specific to normal differentiate granulocyte or monocyte cells.

Nitroblue tetrazolium (NBT) reduction test: The ability to reduce NBT was measured using conventional techniques. Briefly, 2.10⁵ cells were suspended in 900 μl of RPMI 1640 medium and were incubated in the presence of 0.2 μg/ml of 12-O-tetradecanoylephorbol-13-acetate (TPA, Sigma) and 0.5 mg/ml of NBT (Sigma) for 30 minutes at 37° C. The reaction was stopped at 4° C., the cells were cytocentrifuged and subjected to May-Grünwald-Giemsa staining agent. The percentage of cells containing black NBT reduction deposits was determined in duplicate, under an optical microscope, after examining 300 cells.

Flow cytometry analysis of CD14 and CD15 expression: the AML blasts were suspended at a rate of 10⁵ blasts/ml of RPMI 1640 medium containing 0.02% bovine serum albumin and 0.02% 10 cell/ml NaN₃, and were then incubated at 4° C. for 30 minutes in the presence of mAbs conjugated with fluorescein isothiocyanate (FITC), and directed against CD14 (5 μg/ml IgG2b, Coulter Immunology, Hialeah, Fla.) or directed against CD15 (1 μg/ml IgM, Becton Dickinson, San José, Calif.). The mAbs were used at saturation concentrations. The murine IgM and IgG2b conjugated with FITC were obtained from Coulter-Immunotech (Coulter-Immunotech Inc., Westbrook, Me.), and were used at a 1:50 dilution. The bonding of the mAbs directed against CD14 and CD15 was quantified by measuring, by flow cytometry, the cell fluorescence in relation to that of cells labelled with the IgG-FITC. The measurement was made using a FACSvantage (Becton Dickinson) equipped with an INNOVA70-4 argon ion laser (Coherent Radiation, Palo Alto, Calif.) set at 488 nm and operating at 500 mW. The flow cytometer was calibrated using fluorescent beads (Becton Dickinson). This measurement was made on 3000 cells.

Cytological Study:

The cell smears, prepared in triplicate, were stained by May-Grünwald-Giemsa staining and their cytology was examined under an optical microscope.

PML-RARα Oncoprotein Degradation Analysis:

The total cell proteins were extracted from the treated blasts and controls, separated on 8% acrylamide gel in sodium dodecyl sulphate (SDS) and electrotransferred to a nitrocellulose membrane (Laboratoires BioRad). After blocking non-specific fixation sites with 5% skimmed milk in phosphate buffer solution (PBS), the transfers were incubated overnight, in the presence of a 1:2000 dilution of an anti-RARα polyclonal rabbit antibody (Blood 88: 2826-2832, 1996 Raelson et al.). After three washes for 20 minutes in PBS, the fixation of the anti-RARα polyclonal rabbit Ab was detected by incubating with an antirabbit goat antibody labelled with peroxidase, and then by chemoluminescence (ECL detection system, Amersham Life Science, Arlington Heights, Ill.).

Protein Phosphorylation Analysis on Tyrosine Residue

Protein phosphorylation induction on tyrosine residue: HA-12 (50 μg/ml) was added at the time t=0 to 2.10⁵ AML blasts suspended, at ambient temperature, in 200 μl of RPMI 1640 medium containing 10% FCS. At t=1 min, 5 min, 15 min and 30 min, 200 μl of Permeafix Ortho (Coulter-Immunotech Inc., Westbrook, Me.) was added to stop the phosphorylations and permeabilise the cells. After 40 minutes of incubation at ambient temperature, and three washes in PBS, the phosphorylated proteins on the tyrosine residue were labelled specifically with 6D12 mAb (used at 2 μg/ml conjugated with FITC, and the labelling intensity was measured by flow cytometry with reference to the isotype control (cells labelled with IgG1 conjugated with FITC), as described above.

Tyrosine phosphorylation inhibition with genistein: 2.10⁵ AML blasts, suspended in 200 μl of RPMI 1640/10% FCS medium, were incubated in the presence of 50 nM/l of genistein (Calbiochem-Novabiochem, San Diego, Calif.) for 1 hour at 37° C., and then in the presence of HA for either one hour (for studies on cytokine transcript expression), or five days (for studies on differentiation). The Trypan blue exclusion test demonstrated that the treatments are not cytotoxic, the cell viability being greater than 95%.

HA-FITC Bonding:

An HA-FITC preparation was produced with hHA (human hyaluronic acid) and FITC using conventional techniques. The cells were washed three times in phosphate buffer solution (PBS) incubated with 2.5 μg/ml HA-FITC in PBS for 30 minutes on ice, and washed in PBS containing 2% FCS and 0.02% sodium azide (Marking Medium, MM). The HA-FITC bonding was measured by flow cytometry, as described above, with reference to non-labelled cells. To ensure that the labelling observed is specific for HA, the cells were pre-incubated at +4° C. with non-fluorescent HA (100 mg/ml) and abrogation of HA-FITC fixation was detected. The role of CD44 in the HA-FITC fixation was demonstrated by detecting whether anti-CD44 mAbs such as J173 (25 μg/ml) in particular inhibit this fixation.

RT-PCR cytokine transcript expression study: the total RNA was extracted from 5.10⁵ cells using Trizol reagent (Life Technologies, Cergy Pontoise, France), followed by a phenolchloroform extraction and isopropanol precipitation. One microgram of total RNA heated at 70° C. for 10 minutes was used as a matrix for the synthesis of the first strand of complementary DNA (cDNA), by adding reverse transcriptase and random hexamers (Life Technologies). The transcript of the glyceraldehyde phosphodehydrogenase (GADPH) housekeeping gene was used as an internal marker for the PCR reaction (0.24 kb amplification product). Equilibrated quantities of cDNA were used for the PCR amplification of the cytokine transcripts, the primers used for PCR were as follows:

Macrophage Colony Stimulating Factor:

| | |
|---|---|
| M-CSF: | |
| 5′-CATGACAAGGCCTGCGGTCCGA-3′ | (SEQ ID |
| | No. 1) |
| and | |
| 5′-GCCGCCTCCACCTGTAGAACA-3′; | (SEQ ID |
| | No. 2) |

Granulocytic Colony Stimulating Factor:

| | |
|---|---|
| G-CSF: | |
| 5′TTGGACACACTGCAGCTGGACGTCGCCGACTTT-3- | (SEQ ID |
| | No. 3) |
| and | |
| 5′-ATTGCAGAGCCAGGGCTGGGGAGCAGTCATAGT-3′ | (SEQ ID |
| | No. 4) |
| (Genset, Ivry, France). | |

The PCR protocol consisted of 30 cycles of 94° C. for 1 minute, 58° C. for 1 minute and 72° C. for 1 minutes, using a thermocycler (Perkin Elmercetus, Norwalk, Conn.) For each experiment, two negative controls were subject to all the steps. The PCR amplification products (M-CSF: 395 pb, G-CSF: 470 pb) were separated by electrophoresis on 1% agarose gel, and displayed by ethidium bromide staining. In order to demonstrate the specificity of the amplification, the PCR amplification products were transferred to an Immobilon-S membrane (Millipore Inc, France) and hybridised with specific oligonucleotide probes labelled at the 5′ end with ³²P:

| | | |
|---|---|---|
| M-CSF | 5′-TCAGCAAGAACTGCAACAACAGC-3′; | (SEQ ID |
| | | No. 5) |
| G-CSF | 5′GTGAGGAAGATCCAGGGCGA-3′ | (SEQ ID |
| | | No. 6) |
| | (Genset, Ivry, France). | |

### Results and Discussions

### Induction by HA of AML Leukaemic Blast Differentiation

Leukaemic blasts were isolated from blood or bone marrow of patients suffering from different AML sub-types (n=24, Table I) and cultured in the presence hyaluronic acid (HA) for 5 days (see materials and methods). The results obtained are summarised in table I below.

TABLE I
AML blast differentiation induced by HA

| | | Number of cases |
|---|---|---|
| | Number of cases | of |
| AML sub-type | analysed | differentiation |
| AML1/2 | 7 | 5 |
| AML3 | 16 | 12 |
| AML4 | 4 | 3 |
| AML5 | 8 | 6 |
| Total number | 35 | 26 i.e. 74% |

The AML sub-types are defined by French-American-British (FAB) classification criteria. FIG. 1A shows schematically, for the most frequent AML sub-types (AML1/2, AML3, AML4, AML5), the myeloid differentiation stage, the inhibition of which each sub-type corresponds.

The results obtained demonstrate the HA stimulate leukaemic blast differentiation in all AML sub-types (5/7 for AML1/2, 12/16 for AML3, 3/4 for AML4 and 6/8 for AML5). In addition, the range of this differentiation measured by the NBT test was as high for AML3 and AML5 as that obtained after treating AML3 with RA (all-transretinoic acid).

FIG. 1B gives a schematic representation of hyaluronic acid (HA) molecules: human hyaluronic acid hHA containing 2500-5000 disaccharide units, hHA fragment containing 6 disaccharide units (HA-12) and hHA fragment containing 3 disaccharide units (HA-6). Notably, hyaluronic acid forms in the haematopoietic compartment a ligand of the CD44 cell surface molecule.

These AML blast differentiation results were particularly displayed by (see materials and methods):

induction of an ability to reduce nitroblue tetrazolium,

an increased expression of specific line antigens, i.e. specific CD15 for granulocytic differentiation and specific CD14 for monocytic differentiation,

induction of specific cytological characteristics.

Firstly, the ability to produce an oxidation-reduction reaction was analysed using the nitroblue tetrazolium (NBT⁺) reduction test. Table II below illustrates the percentages of NBT positive blasts observed among the blasts from AML1/2, AML3, AML4, AML5 patients, after HA treatment or after negative control treatment as described in the materials and methods (for AML3: positive control treated with RA).

TABLE II
Induction of ability to reduce nitroblue tetrazolium

% NBT⁺ cells (extreme values)

| AML Type | negative controls | treated with HA |
|---|---|---|
| M1/M2 | <5 | 7-23 |
| M3 | <5 | 20%-80% |
| | | (RA: 47-90) |
| M4 | 10%-38% | 43%-90% |
| M5 | <5 | 32%-70% |

As expected in the negative control groups, less than 5% of the blasts from AML1/2, AML3 and AML5 are observed as NBT⁺. In contrast, after incubation in the presence of HA (see materials and methods), the proportion of NBT⁺ blasts increased significantly for all the sub-types, indicating that they differentiate. Indeed, in 12 out of 16 cases of AML3, the % of NBT⁺ cells is between 20% and 80% (median value 42%). In addition, in 6 of them, the % observed is as high as that obtained with all-transretinoic acid treatment (over 50% of cells are NBT⁺). Similarly, in 6 out of 8 cases of AML5, the % of NBT⁺ cells is between 32% and 70% (median value 52%). In 5 out of 7 cases of AML1/2, the % of NBT⁺ cells increased to reach 20-25% (median value 22%). This value is significantly greater than that observed in the negative controls, but lower than those observed in the cases of AML3 and AML5, indicating that the maturation of particularly immature blasts (AML1/2) was, at the doses and times applied in this case, more limited than in the cases of AML3 and AML5. For AML4, which are cases of more mature blasts, 10% to 38% (median value 18%) of the negative controls were observed as NBT⁺. After treatment, this proportion increases to reach 43% to 90% (median value 55%, i.e. 3 times higher than in the controls). These results indicate that CD44 activating molecules such as hyaluronic acid, or its fragments up to HA-6, induce the differentiation of AML blasts from all the sub-types.

Secondly, we measured by flow cytometry, the level of expression of specific line antigens on AML blasts.

The expression of CD15 was used to monitor the differentiation of AML3 blasts (promyelocytic sub-type) since it is specific for the granulcytic line.

TABLE III
Increase in the percentage of cells expressing
CD14 (monocytic) and/or CD15 (granulocytic)
differentiation antigens

| | % CD14⁻ cells | | % CD15⁻ cells | |
|---|---|---|---|---|
| | median values | | median values | |
| | (extreme values) | | (extreme values) | |
| | | treated | | treated |
| AML type | controls | with HA | controls | with HA |
| M1/M2 | <10% | (42-100) | (0-56) | 22-100 |
| M3 | — | — | (69) 28-87 | 78 (42-90) |
| M5 | (<5) | (50-91) | — | — |

Table III above illustrates the percentages of CD15 positive and CD14 positive blasts measured by flow cytometry, as described in the materials and methods, among the blasts treated with HA or the negative control blasts which were stained with antibodies conjugated with FITC directed against CD (specific granulocytic process antigen) or against CD14 (monocytic process antigen). These percentages were determined with reference to isotype controls.

Changes in the MFI (mean fluorescence intensity, arbitrary units) are illustrated in FIG. 2A, which represents the number of CD14⁺ and CD15⁺ cells induced using HA on AML1/2, 3, 4 and 5, as a function of the fluorescence intensity (FITC), from left to right, and from top to bottom: AML1/2 CD14 graph, AML1/2 CD15 graph, AML3 CD15 graph, AML4 CD14 graph, AML4 CD15 graph, AML5 CD14 graph (black curves: use of HA; grey curves, further to the left: negative controls).

AML3:

In the negative controls, CD15 was moderately expressed (range of mean fluorescence intensity (MFI) values: 7 to 223) among 28 to 87% of AML3 blasts (mean value 69%, except for 2 samples found to be CD15 negative). After treatment with HA (CD44 activation), the % of CD15⁺ cells increased in 12 out of 16 samples to reach values of 42 to 90% (median value 78%), as high as which all-transretinoic acid administered to 8 cases. In addition, the MFI values increased approximately 3 fold (see FIG. 2A) and are as high as, or higher than, with all-transretinoic acid.

AML5:

For six AML5 samples, CD14 was not detectable in the negative controls: in five of these samples, up to 50%-91% of leukaemic cells were found to be CD14+ after treatment with HA (CD44 activation), see table III. In addition, two other AML5 samples showed significant quantities of CD14 in the negative controls: in one of these two samples, the level of CD14 was increased significantly after treatment with HA (CD44 activation), with an MFI value of up to 340 compared to 102 in the negative controls (see FIG. 2A, bottom right graph).

These results indicate that AML3 and AML5 blasts mature to granulocytic and monocytic lines, respectively. For 8 of the 12 cases of AML3 (66%), the maturation was as marked as after treatment with all-transretinoic acid.

AML1/2:

The expression of CD14 and that of CD15 were measured on AML1/2 since these very immature myeloblastic leukaemic cell sub-types may have retained the ability to differentiate to two granulocytic and monocytic lines, like normal immature myeloid progenitor cells. The expression of both CD14 and CD5 increased for 6 of the 8 cases of AML1/2 after HA treatment (CD44 activation). For both CD14 and CD15, the proportion of cells expressing these differentiation antigens increased: proportion of CD14⁺ positive cells: less than 10% in the controls, 42% to 100% in the treated groups: proportion of CD15⁺ positive cells: 0% to 56% in the controls, 22% to 100% in the treated groups (see table III). The expression intensity of these antigens (mean fluorescence intensity) also appears to have increased in relation to the MFI values which multiplied by a factor of around 2 (see FIG. 2A).

AML4:

Finally, the AML4 blasts which spontaneously show granulo-monocytic phenotype characteristics also differentiate along the monocytic and granulocytic lines, as demonstrated by the increase in the % of cells expressing the CD14 and CD15 differentiating antigens (% of positive cells multiplied by a factor of 2, see table III) and the increase in the MFI values which are multiplied by a factor of 3 (see FIG. 2A).

In this way, as demonstrated by the NBT reduction test, the measurement of the expression of CD14 and/or CD15 indicates that the bonding of CD44 with activating molecules such as hyaluronic acid induces the differentiation of all AML blast sub-types.

Thirdly, the induction of specific cytological characteristics for mature cells was studied. These results are illustrated in FIG. 2B which shows six May-Grünwald-Giemsa stainings, from left to right, top photos: negative controls on AML3, AML3 blasts treated for 5 days with HA, AML3 blasts treated with RA (positive controls); bottom photos: negative controls on AML5, AML5 blasts treated with HA, AML1 blasts treated with HA (NBT⁺ cells).

In the case of AML3 (FIG. 2B, top line), the negative controls (untreated blasts at far left) show an immature promyelocytic phenotype characterised by a high nucleo-cytoplasmic ratio, numerous nucleoles and abundant azurophilic cytoplasmic granulations: Auer bodies which are typical of M3 AML are observed (arrow). After HA treatment (FIG. 2B, centre and right photos, top line), the cases of AML3 show a segmented nucleus, a low nucleo-cytoplasmic ratio, rare nucleoles, some azurophilic granulations, which are typical of differentiated granulocytic cells (band cells and metamyelocytes). These characteristics are similar to those of the blasts treated with all-transretinoic acid (right photo, top line), which form the positive control for AML3 differentiation. Cytoplasmic structures resembling damaged Auer body structures may be observed (arrow).

In the case of the AML5 blasts (FIG. 2B, bottom line), the negative controls (photo on left) show a high nucleo-cytoplasmic ratio, chromatin finely cross-linked with numerous molecules and a regular shape, characteristic of immature monoblastic cells. After HA treatment (CD44 activation) (FIG. 2B, bottom line, centre photo), the AML5 blasts show a decrease in the nucleus/cytoplasm ratio, a decrease in the number of nucleoles, chromatin condensation, and irregular cytoplasmic contours, all these characteristics being typical of mature monocytes.

In the case of AML1 (FIG. 2B, bottom line, photo on right), the NBT⁺ cells after HA treatment are easily recognised by the dark cytoplasmic staining due to NBT reduction (×100).

Therefore, the cytological examination demonstrates that the AML3 and AML5 blasts differentiate up to the terminal granulopoiesis and monopoiesis stages, respectively, after HA treatment (CD44 activation). Indeed, after HA treatment, the AML3 blasts show a segmented nucleus, some nucleoles and a restricted number of azurophilic granulations. These cytological characteristics, which are similar to those observed after treatment with all-transretinoic acid are characteristic of differentiated granulocytic cells (metamyelocytes and segmented polymorphous cells). In addition, the AML5 show, after HA treatment, a decrease in the nucleus/cytoplasm ratio, a decrease in the number of nucleoles, chromatin condensation, and show irregular cytoplasmic contours, all these characteristics being typical of mature monocytes. These cytological characteristics corroborate the previous observations made for AML3 and AML5 according to functional and antigenic differentiation criteria. No cytological change was observed in the AML1/2 after HA treatment, according to the procedure, since AML1/2 blasts are very immature: their terminal differentiations require more than 6 days of incubation and/or the action of other differentiating molecules such as cytokines to complete their differentiation up to the terminal stage in vitro.

HA Modes of Action

We also demonstrate that the intensity of the differentiation is directly related to the dose of activating molecules used, and to the incubation time applied. FIG. 3A illustrates the results obtained by incubating, as described in the methods, AML5 blasts in the presence of the specified concentrations of HA-12 (FIG. 3A, left graph), or in the presence of 15 μg/ml of HA-12 for 3, 5 and 6 days (FIG. 3A, right graph). The data represents the mean fluorescence intensity (MFI+standard deviation) of samples produced in triplicate and taken from a representative element of three experiments.

Therefore, the differentiation intensity induced by HA is related to the dose of activating molecules used: up to 15 μg/ml of HA, in the case of AML5, as demonstrated in FIG. 3A.

In addition, the ability of CD44 ligands, and of anti-CD44 monoclonal antibodies in particular, to significantly inhibit the bonding of hHA with AML blasts confirms that this hHA bonding is at the very least performed via CD44. This is illustrated in FIG. 3B which represents, as a function of the log fluorescence intensity, and identifying the curves from left to right for each graph, the number of negative control cells and cells treated with HA-FITC only (left graph), the number of negative control cells, cells treated with unlabelled HA and then treated with HA-FITC and cells treated with HA-FITC only (centre graph), and the number of negative control cells, cells treated with anti-CD44 mAbs followed by HA-FITC, and cells treated with HA-FITC only (right graph).

The differentiating ability of different anti-CD44 monoclonal antibodies (mAb) was tested in vitro on cells sampled from AML patients as for HA. All the activating anti-CD44 mAbs tested proved to be incapable of inducing AML blast differentiation under these conditions: these include the murine mAb, Hermes 1, which proved to be ineffective when used alone. However, other activating anti-CD44 mAbs proved to have an equivalent efficacy to hyaluronic acid: the murine mAb F10-44-2, for example. In addition, it may be noted that, using anti-CD44 mAb with differentiating activity, it is possible to produce, using conventional techniques, products wherein the activity is comparable to that of HA, of HA fragments (HA6-HA20). When these products are mAbs of non-human origin, it may for example prove to be advantageous to humanise them (grafting of CDR, Fab or (Fab′)₂ fragments onto a human matrix antibody, for example) in order to prevent antigenic reactions.

In relation to the mechanisms by means of which HA exerts its differentiating action, it was noted that, remarkably, anti-CD44 mAbs with differentiating activity produce a cross-reaction with HA on CD44, unlike anti-CD44 mAbs with non-differentiating activity. These different results demonstrate the existence on CD44 of at least one epitope specifically involved in myeloid differentiation. This epitope related to differentiation is located inside the HA to CD44 bonding domain. It can be identified by those skilled in the art using ELISA tests, after enzyme digestion of CD44 in peptide fragments. The oligonucluotide sequence (SEQ ID No.7, sequence between two square brackets, top lines), delimited by two arrows, of said bonding domain of HA on CD44 and its peptide sequence (SEQ ID No. 8, sequence between two square brackets, bottom lines) are given below.

```
CAAGTTTTGGTGGCACGCAGCCTGGG [ACTCTGCCTCGTGCCGCTGAGCCTGGCGCAGATCGATTTGAATATAAC
  K F W W H A A W G [ L C L V P L S L A Q I D L M I T
                                                                                                                                        --CHO--
CTGCCGCTTTGCAGGTGTATTCCACGTGGAGAAAAATGGTCGCTACAGCATCTCTCGGACGGAGGCCGCTGACCTCTG
  C R F A G V F H V E K N G R Y S I S R T E A A D L C
CAAGGCTTTCAATAGCACCTT GCCCACAATGGCCCAGATGGAGAAAGCTCTGAGCATCGGATTTGAGACCTGCAG
  K A F N S T L P T M A Q M E K A L S I G F E T C R
                    --CHO--
GTATGGGTTCATAGAAGGGCATGTGGTGATTCCCCGGATCCACCCCAACTCCATCTGTGCAGCAAACAA] CAC
  Y G F I E G X V V I P R I H P N S I C A A N N ] T
                                                                                                                                  --CHO--
AGGGGTGTACATCCTCACATACAACACCTCCCAGTATGACACATATTGCTTCAATGCTTC
  G V Y I L T Y N T S Q Y D T Y C F N A S
                                            --CHO-- --CHO--
```

It was also observed that some differentiating anti-CD44 mAbs, which they are used in the presence of hHA or hHA fragments, are capable of stimulating bonding of hHA (or an hHA fragment) with its target, while others, on the other hand, block and inhibit the differentiating effect (this is particularly the case for the mAb J173).

It was also observed that non-differentiating anti-CD44 mAbs are capable of inhibiting bonding of HA with CD44. This is the case for example of Hermes 1. This result suggests that the recognition by HA of a specific epitope on CD44 may not be sufficient to induce the differentiation observed.

In addition, in a minority of cases of AML (9/36), it was not possible to induce differentiation by CD44 ligands such as HA. In these cases, the flow cytometry analysis demonstrated that the monoclonal antibodies which produce a cross-inhibition with HA do not bond with CD44. However, CD44 was expressed on these blasts, since this molecules was labelled with anti-CD44 antibodies conjugated with FITC. These results suggest that the accessibility of the epitope(s) involved in differentiation could be prevented by a particular conformation of the CD44 protein, or by particular glycosylation patterns on the CD44 molecule, as observed in several cases of AML.

Therefore, in addition to one or more CD44 sequences involved in differentiation, for a normal differentiation sequence, conformation constraints of this molecule are also involved.

### Molecular Events

To go further in the molecular events related to differentiation induced by CD44, the degradation of the PML-RARα oncoprotein in AML3 blasts under the effect of HA was studied, as reported with all-transretinoic acid. For this purpose, protein extracts from AML3 blasts (treated with HA, treated with RA or negative control blasts) were subjected to electrophoresis, transferred and confronted with a specific anti-RARα monoclonal antibody as described in the materials and methods. The results are illustrated in FIG. 4A on which the 110 kD band corresponding to PML-RARα (negative control blasts) appears to be significantly reduced 24 hours after CD44 activation by HA (track 2), i.e. as effectively as with RA (all-transretinoic acid, track 3), while the wild type RARα protein (band at approximately 50 kD) does not change.

It was also demonstrated that the differentiation induced by CD44 involves

1) tyrosine phosphorylations, and

2) in several cases, but not in all, the induction of the expression of cytokine messengers, i.e. key events of normal granulomonocytic differentiation.

Firstly, the inventors demonstrated that the phosphorylation of proteins on tyrosines is crucial in the AML3 and AML5 differentiation induced by CD44 (HA treatment), since genistein, a specific tyrosine kinase inhibitor, inhibits this differentiation. To corroborate this result, the inventors demonstrated, using an antibody (6D12) conjugated with FITC and flow cytometry as described in the methods, that intracellular tyrosine phosphorylations are already induced after one minute of treatment. This is illustrated in FIG. 4C which represents the mean fluorescence intensity of phosphorylated tyrosines as a function of time, for blasts treated with HA (top curve: 1 representative case of AML5), in comparison to negative control blasts (bottom curve).

Secondly, the following cytokines are known to be specific players in the induction of normal granulo-monocytic differentiation: GM-CSF (Granulomonocytic Colony Stimulating Factor), G-CSF and M-CSF. Using the semi-quantitative polymerase chain reaction with reverse transcriptase (RT-PCR), 1 hour after the activation of CD44 (HA treatment), M-CSF transcripts are detected in M1/M2 AML (1 out of 3 cases) and an M-CSF transcript is detected in M5 AML (1 out of 3 cases). This is illustrated in FIG. 4B which represents agarose gels obtained after ethidium bromide staining of AML5 blasts (photos on left: top gel M-CSF, bottom gel: GAPDH marker; track 1: controls, track 2: treated with HA).

Therefore, these differentiation inductions involved tyrosine phosphorylations, since they are abrogated by the treatment with genistein. It is important to note that in numerous cases of AML, GM-CSF, G-CSF and M-CSF transcripts were either not detected or expressed in a constitutive manner. This suggests that these cytokines are not necessarily involved in the differentiation induced by CD44 (HA treatment).

In conclusion, the inventors demonstrated that AML blast differentiation may be induced and/or stimulated by HA or its fragments (from HA-6), particularly via CD44, for all AML sub-types. In AML3, the differentiation induced by HA is comparable to that obtained with retinoic acid. The results given particularly enable the development of new therapies for AML differentiation, particularly for all the M1 to M5 sub-types, using hyaluronic acid structure molecules, and/or the targeting of the CD44 molecule with other agonist molecules.

### EXAMPLE 2

### Production of a Medicinal Product Intended to Stimulate or Induce Human Haematopoietic Cells

The hyaluronic acid (HA) used in example 1 above was purified from human umbilical cord (ICN Pharmaceuticals, Sigma). For the industrial production of medicinal products, HA may also be purified from non-human tissue: cockscomb (produced by the company Pharmacia under the trade name Healon) or streptococcus, for example. These HA molecules have a high molecular weight. However, it is small HA molecules, which may particularly be obtained by enzyme digestion of the high molecular weight form, which offer the best differentiating properties according to the invention. A use according to the invention advantageously comprises the use of “HA fragment” molecules composed of 3 to 10 disaccharides (HA-6 to HA-20, see FIG. 1B). The use of these small molecules also offers an advantage for pharmaceutical production, since they are less likely to be trapped by the liver than high molecular weight HA molecules.

Any pharmaceutical form may be envisaged for the medicinal product according to the invention. As HA is very water-soluble, a preparation in dissolved form in equilibrated saline solution may be easily produced.

As haematopoietic tissue (bone marrow, spleen, lymph glands) show a strong affinity for HA, such a solution may be effectively administered by injecting by the intravenous route. Administration doses of the order of 1 to 10 mg of HA/kg, advantageously of the order of 2 to 5 mg of HA/kg, and particularly of the order of 3 mg of HA/kg appear to be advantageous.

If necessary, and particularly in view of the follow-up results of the progression of the disease in the patient, the doses may be increased (in a unit dose) and/or repeated (over time): HA offers the considerable advantage of not being toxic and thus enabling an administration dosage which is perfectly adapted to the patient in question.

Finally, it may be beneficial to reduce the significant fixation of HA on the liver sinuses. In this organ, HA is fixed by the ICAM1 surface molecule, and not by CD44. The preventive inhibition of this fixation may be provided for by injecting chondroitin sulphate which would saturate the ICAM-1 receptor sites.

The ability of CD44 to fix HA is variable, sometimes low in the constitutive state, but can be considerably activated by certain activating type anti-CD44 monoclonal antibodies (MAb) (see example 1). For this reason, such MAbs may, particularly advantageously, be incorporated in the medicinal product according to the invention as adjuvant(s) of the differentiation induced by HA. Therefore, it is possible to envisage injecting them at the same time as HA. On the basis of the monoclonal antibody doses currently used in AML cytotoxic therapy, doses of the order of 5 to 10 mg/m² of activating anti-CD44 MAbs appear to be indicated.

### EXAMPLE 3

### Differentiation Action of Hyaluronic Acid on Normal Human Bone Marrow CD34⁺ Haematopoietic Progenitors

### Materials and Methods

The CD34⁺ haematopoietic cells are isolated from normal human bone marrow by immunoadsorption on magnetic beads coated with anti-CD34 antibodies. These cells are inoculated at a rate of 500 cells/200 μl in serum-free culture medium (Stemcell medium) supplemented with the cytokines IL-1 (100 U/ml), IL-3 (2 ng/ml) and SCF (10 ng/ml) and 50 μg/ml of HA (molecules composed of 10 to 50 saccharides). HA is not added to the control groups. After 7 days of incubation at 37° C., the expression of the CD15 (granulocytic) and CD14 (monocytic) differentiation antigens is analysed by flow cytometry. The same experiment is carried out with CD34+ haematopoietic cells isolated from umbilical cord blood.

### Results

The number of CD15⁺ and CD14⁺ cells in the different treatments is measured by fluorescence intensity. The results obtained for CD34⁺ haematopoietic cells isolated from bone marrow are illustrated in FIG. 5. The results obtained for haematopoietic cells isolated from umbilical cord blood are comparable. In both cases, a significantly greater proportion of CD15⁺ and CD14⁺ cells are observed in the groups treated with HA fragments.

### Conclusion

HA fragments stimulate the differentiation of CD34⁺ haematopoietic progenitor cells isolated from human bone marrow, i.e. from very immature strain cells (CD15⁻, CD14⁻), not only according to the monocytic process, but also according to the granulocytic process.

### EXAMPLE 4

### HA Fragments Comprising More Than 10 Disaccharide Units

Using a procedure comparable to that in example 1, it can be observed that we demonstrated that HA fragments composed of 20 to 100 saccharide units and used at a rate of 50 μg/ml induce terminal differentiation of AML1 and AML2 blasts. This differentiation is demonstrated:

by the increase in the expression of CD14 and CD15 differentiation antigens:

AML1:

13% CD14⁻ cells in the treated group compared to less than 5% in the control group, 72% CD15⁺ cells (relative fluorescence intensity of 56) in the treated group compared to 55% CD15⁺ (relative fluorescence intensity of 21) in the control group.

AML2:

35% CD14⁺ cells in the treated group compared to less than 5% in the control group.

by the induction of NBT⁺ cells: 50% in the treated group (AML1) compared to less than 5% in the control group.

by the induction of specific cytological characteristics for mature monocytes.

1. An in vitro method of regulating the differentiation of hematopoietic cells comprising contacting said hematopoietic cells with a differentiation regulating amount of a composition comprising a polymer, said polymer comprising disaccharide units each comprised of an N-acetyl D-glucosamine structure molecule bonded by an O-glycoside β1,4 bond with a glucouronic acid structure molecule, such that differentiation of said hematopoietic cells are regulated.

2. A method of treating a person suffering from at least one of leukaemia, aplasia and neutropenia, said method comprising administering to said person a hematopoietic cell stimulating amount of a composition comprising a polymer, said polymer comprising disaccharide units each comprised of a N-acetyl D-glucosamine structure molecule bonded by an O-glycoside β1,4 bond with a gluceuronic acid structure molecule, such that said hematopoietic cells are stimulated or induced to differentiate.

3. The method of claim 1 wherein said hematopoietic cells are CD14 negative/CD15 negative cells.

4. The method of claim 2 wherein said hematopoietic cells are CD14 negative/CD15 negative cells.

5. The method of claim 1 wherein said hematopoietic cells are stimulated or induced to differentiate upon said contacting.

6. The method of claim 3 wherein said hematopoietic cells are stimulated or induced to differentiate upon said contacting.

7. The method of claim 1 wherein said polymer is a mimetic of hyaluronic acid.

8. The method of claim 2 wherein said polymer is a mimetic of hyaluronic acid.

9. The method of claim 3 wherein said polymer is a mimetic of hyaluronic acid.

10. The method of claim 4 wherein said polymer is a mimetic of hyaluronic acid.

11. The method of claim 1 wherein said composition further comprises an adjuvant involved in myeloid differentiation.

12. The method of claim 2 wherein said composition further comprises an adjuvant involved in myeloid differentiation.

13. The method of claim 11 wherein said adjuvant is an anti-CD44 antibody or CD44-binding antibody fragment.

14. The method of claim 12 wherein said adjuvant is an anti-CD44 antibody or CD44-binding antibody fragment.

15. The method of claim 1 wherein said regulating is accomplished in the absence of exogenous cytokine.

16. The method of claim 2 wherein said regulating is accomplished in the absence of exogenous cytokine.

17. The method of claim 1 wherein said polymer comprises at least 3 disaccharide units.

18. The method of claim 2 wherein said polymer comprises at least 3 disaccharide units.

19. The method of claim 2, further comprising administering to said person at least one inhibitor agent which binds ICAM1 and inhibits binding of ICAM1 to said polymer.

20. The method of claim 1 wherein said hematopoietic cells are leukaemic cells.

21. The method of claim 2 wherein said hematopoietic cells are leukaemic cells.

22. The method of claim 20 wherein said cells are any one of AML1/2, AML3, AML4 and AML5 blasts.

23. The method of claim 21 wherein said cells are any one of AML1/2, AML3, AML4 and AML5 blasts.

24. A medicinal product intended to induce or stimulate the differentiation of cells selected from the group consisting of leukaemic cells and CD14 negative/CD15 negative stem cells, said product comprising a polymer comprising an effective quantity of disaccharide units each composed of an N-acetyl-D-glucosamine structure molecule bonded by an O-glycoside β1, 4 bond with a glucuronic acid structure molecule, and anti-ICAM1 monoclonal antibody or an ICAM1 binding fragment thereof.

25. A method of producing a medicinal product intended to induce or stimulate the differentiation of cells selected from the group consisting of leukaemic cells and CD14 negative/CD15 negative stem cells, said method comprising admixing in said medicinal product a polymer comprising an effective quantity of disaccharide units each composed of an N-acetyl-D-glucosamine structure molecule bonded by an O-glycoside β1, 4 bond with a glucuronic acid structure molecule, and an adjuvant involved in myeloid differentiation, wherein said adjuvant comprises an anti-CD44 antibody or a CD44-binding fragment thereof.

26. A medicinal product intended to induce or stimulate the differentiation of cells selected from the group consisting of leukaemic cells and CD14 negative/CD15 negative stem cells, said medicinal product comprising a polymer comprising an effective quantity of disaccharide units each composed of an N-acetyl-D-glucosamine structure molecule bonded by an O-glycoside β1, 4 bond with a glucuronic acid structure molecule, and an adjuvant involved in myeloid differentiation.

27. The medicinal product of claim 26 wherein said adjuvant comprises an anti-CD44 antibody or a CD44-binding fragment thereof.

28. A method of claim 1 wherein said polymer is a hyaluronic acid polymer or a fragment thereof.

29. A method of claim 2 wherein said polymer is a hyaluronic acid polymer or a fragment thereof.

30. A product of claim 24 wherein said polymer is a hyaluronic acid polymer or a fragment thereof.

31. A product of claim 26 wherein said polymer is a hyaluronic acid polymer or a fragment thereof.

32. A product of claim 27 wherein said polymer is a hyaluronic acid polymer or a fragment thereof.

33. A method for predicting the therapeutic benefit of a medicinal product intended to induce or stimulate the differentiation of cells selected from the group consisting of leukaemic cells and CD14 negative/CD15 negative stem cells, said method comprising placing in contact under physiological conditions leukaemic blasts from a patient and said medicinal product, determining whether said blasts differentiate in vitro, and predicting from said determination whether said product will be therapeutically beneficial to said patient.
